(19) United States
(12) Patent Application Publication
(10) Pub. No.: US 2006/0171910 A1
(43) Pub. Date: Aug. 3, 2006

(54) Cosmetic composition containing an alkoxylated alcohol ester and a hydrocarbon-based ester oil

(76) Inventors: Audrey Ricard, Paris (FR); Caroline Lebre, Thiais (FR); Vanina Filippi, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/320,663

(22) Filed: Dec. 30, 2005

Related U.S. Application Data
(60) Provisional application No. 60/642,993, filed on Jan. 12, 2005.

(30) Foreign Application Priority Data
Dec. 30, 2004 (FR) 0453265

(51) Int. Cl. A61K 8/00

(52) U.S. Cl. 424/70.1; 510/530

(57) ABSTRACT

The present disclosure relates to a composition comprising at least one ester of an alkoxylated alcohol and a carboxylic acid and at least one ester oil different from the preceding ester, the composition being capable of forming a deposit with a staying power index of greater than or equal to 30%. The alkoxylated alcohol ester may especially be octyldodecyl PPG-3 myristyl ether dimer dilinoleate. This composition may be used as a care and/or makeup product for keratin materials, especially for the skin, the lips and/or the integuments.

This application claims benefit of U.S. Provisional Application No. 60/642,993, filed Jan. 12, 2005, the contents of which are incorporated herein by reference. This application also claims benefit of priority under 35 U.S.C. § 119 to French Patent Application No. 04 53265, filed Dec. 30, 2004, the contents of which are also incorporated herein by reference.

The present disclosure relates to a cosmetic composition containing an alkoxylated alcohol ester, for example, a cosmetic composition for making up or caring for human facial or body skin, the scalp, the lips or the integuments, such as the hair, the eyelashes, the eyebrows or the nails.

The composition of the present disclosure may be a makeup product for the lips, the body or the integuments that may also have care properties. The composition of the present disclosure may be, for example, a lipstick or a lip gloss, a makeup rouge, an eyeshadow, a tattoo product, a mascara, an eyeliner, a nail varnish, an artificial tanning product for the skin, a hair coloring product or a haircare product.

The cosmetic composition according to the present disclosure may exhibit very satisfactory color fastness, while at the same time being glossy and comfortable.

Staying power is desired by users of cosmetic products. Lipsticks with satisfactory staying power are generally sticks containing low molecular weight ester oils or silicone oils. To increase the staying power of these products, the latest formulation concepts are based on the combination of a “glossy” phase and a “color and comfort” phase that are made compatible by means of a volatile solvent. During the application of the product to the lips, the volatile material evaporates and a segregation phenomenon then takes place. This dual phase concept remains, however, limited for improving staying power and comfort. The reason for this is that the field of compatibility of the phases is often restricted and the isododecane content cannot be increased without detriment to the hardness of the sticks and the overall comfort of the composition.

Formulations have been proposed containing acrylate/acrylic copolymers dispersed in a volatile solvent such as isododecane. These polymers form a film on the lips after evaporation of the solvent. However, very high levels of volatile materials are needed in these formulations in order to ensure the staying power of the cosmetic composition on the keratin materials, thus, making the deposit formed by the composition uncomfortable.

It has been found that an alkoxylated ester makes it possible to obtain formulations with high gloss whose comfort is equivalent to that of a standard formulation, while at the same time improving the color fastness as compared to known formulations.

Certain alkoxylated esters have already been used in cosmetic compositions. For example, United States Patent Application Publication No. 2002/0 192 249 describes cosmetic compositions comprising an ester of a monocarboxylic acid containing from 4 to 24 carbon atoms, and of an alcohol comprising a polypropoxyl group and an alkyl chain containing from 2 to 24 carbon atoms. These compositions may include, besides the alkoxylated ester, mineral oil or liquid paraffin. Further, this patent application publication also describes an anhydrous composition containing this ester and a film-forming agent, and a composition containing PPG-3 myristyl ether neoheptanoate. More specifically, this patent application publication describes lipstick compositions containing this polypropoxylated ester in combination with hydrogenated polyisobutene; cream foundation compositions as an emulsion containing this ester; sunscreen compositions as an emulsion containing hexyl laurate, octyl palmitate and cetyl palmitate in combination with this ester.

U.S. Pat. No. 5,693,316 discloses cosmetic compositions containing an alkoxylated fatty ester obtained from a dicarboxylic acid containing from 2 to 22 carbon atoms, such as maleic acid, and a stoichiometric excess of at least one polyalkoxylated fatty alcohol comprising an alkyl chain containing from 14 to 22 carbon atoms and a polyalkoxyl group. These compositions may comprise mineral oil or liquid paraffin as a second emollient. This patent also describes an anhydrous composition containing this ester and a film-forming agent. In one embodiment, the alkoxylated ester is di-PPG-3 myristyl maleate.

U.S. Pat. No. 6,476,254 discloses cosmetic compositions containing an ester of a dicarboxylic acid containing from 4 to 12 carbon atoms and of a polyalkoxylated fatty alcohol in which the non-alkoxylated portion contains from 8 to 36 carbon atoms. The ester may be di-PPG-3 myristyl adipate. The composition may be anhydrous, and may contain mineral oil or liquid paraffin.

International Patent Application Publication No. WO 2003/013 439 relates to esters of C₃-C₂₁ dicarboxylic acids or of C₄-C₂₂, for example, C₃ to C₉ aliphatic tricarboxylic acids and of polyalkoxylated fatty alcohols comprising a C₆-C₃₀, for example, C₁₈-C₂₂ alkyl radical. This patent publication describes a cosmetic composition that may contain petroleum jelly, mineral oil, esters of aliphatic carboxylic acids and of aliphatic alcohols containing from 18 to 40 carbon atoms, a film-forming agent, or a fatty alcohol such as cetyl alcohol.

U.S. Pat. Nos. 5,302,377, 5,455,025 and 5,597,555 disclose cosmetic compositions containing an alkoxylated fatty ester of a tricarboxylic acid, such as citric acid, with a stoichiometric excess of at least one polyalkoxylated fatty alcohols with emollient properties, for topical preparations. These preparations contain a mineral oil as second emollient. These patents also describe the combination of this ester with a film-forming agent. In one embodiment, the ester is tri-PPG-3 myristyl citrate.

International Patent Application Publication No. WO 2004/052 076 describes cosmetic compositions containing mixed esters of polyalkoxylated alcohols and of monohydric alcohols with polycarboxylic acids, such as dicarboxylic acids. These compositions may contain a second emollient such as mineral oil or petroleum jelly. The mixed esters described may be formulated in combination with a film-forming compound.

One embodiment of the present disclosure is a cosmetic composition comprising at least one ester of an alkoxylated alcohol and of a carboxylic acid and at least one hydrocarbon-based ester different from the preceding ester, wherein when said composition forms a deposit, the deposit exhibits a staying power index of greater than or equal to 30%.

Another aspect of the present disclosure is a cosmetic process for preparing a film of cosmetic composition having gloss, staying power and comfort properties, comprising providing a cosmetic composition comprising at least one ester of an alkoxylated alcohol and of a carboxylic acid and at least one hydrocarbon-based ester different from the preceding ester, and applying said the composition to a substrate, such as the lips.

Another aspect of the present disclosure is the use of the combination of at least one ester of an alkoxylated alcohol and of a carboxylic acid and of at least one hydrocarbon-based ester different from the preceding ester, to obtain a cosmetic composition that has gloss, staying power and comfort properties.

A further aspect of the present disclosure is a care and/or makeup process for keratin materials, comprising providing a composition comprising at least one ester of an alkoxylated alcohol and of a carboxylic acid and at least one hydrocarbon-based ester different from the preceding ester, said composition being capable of forming a deposit with a staying power index of greater than or equal to 30%, and applying the composition to keratin materials.

As used herein, the term “alkoxylated alcohol” means a hydrocarbon-based compound comprising at least one —OH function, for example, a hydrocarbon-based compound comprising one —OH function, and at least one group of the formula: 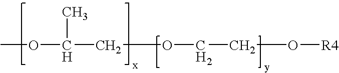
in which x and y are, independently, integers ranging from 0 to 40, the sum of x and y ranges from 1 to 80, and R₄ is an aliphatic or aromatic, saturated or unsaturated, substituted or unsubstituted hydrocarbon-based unit containing from 1 to 36 carbon atoms. In a non-limiting embodiment, R₄ may contain from 4 to 36 carbon atoms.

The alkoxylated alcohol may be a polyalkoxylated alcohol, such that x and y in the above formula are, independently, integers ranging from 0 to 40, and the sum of x and y ranges from 2 to 80. In a non-limiting embodiment, x and y may be, independently, integers ranging from 0 to 30, wherein the sum of x and y is between 2 and 30 inclusive.

The above formula schematically illustrates all the ethoxy units in a first group and all the propoxy units in another group. These units may be placed in any order. For example, these units may be placed randomly, in blocks, or as alternating units. For example, the ethoxy units (E) and the propoxy units (P) of the alkoxylated alcohol may be arranged in the following manner: EEEP, EEPE, EPEE, PEEE, EEEPEPPPE, PEPPPEEEEPE and in similar arrangements. These exemplary arrangements, however, are not limiting.

Alkoxylated Ester

The composition according to the present disclosure contains an alkoxylated ester derived from at least one alkoxylated alcohol and a carboxylic acid. Non-limiting examples of alkoxylated esters that are suitable for use in the present disclosure include:
the esters obtained by reacting a monocarboxylic acid with an alkoxylated alcohol,
the polyesters obtained by reacting a polycarboxylic acid with a stoichiometric excess of at least one alkoxylated alcohol relative to the number of acid functions in the said acid,
polyesters in which only one ester function is obtained by reacting an acid function of a polycarboxylic acid with an alkoxylated alcohol,
polyesters comprising at least one ester function obtained by reacting an acid function of a polycarboxylic acid with an alkoxylated alcohol, and at least one ester function obtained by reacting another acid function of the said polycarboxylic acid with a fatty alcohol,
and mixtures thereof.
   Alkoxylated Ester of Monocarboxylic Acid

The alkoxylated ester may also be chosen from esters of monocarboxylic acids and mono- or poly-alkoxylated fatty alcohols. In a non-limiting embodiment of the present disclosure, the alkoxylated ester is chosen from esters formed by reacting an aliphatic or aromatic monocarboxylic acid with a stoichiometric excess of a polyalkoxylated fatty alcohol, for example, a polypropoxylated alcohol.

In a further non-limiting embdoiment, the alkoxylated ester of a monocarboxylic acid may be chosen from the polypropoxylated monoesters having the following structural formula: 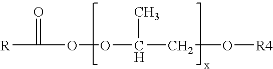
in which x is an integer ranging from 2 to 40, for example, from 3 to 30, more specifically from 3 to 10,

R₄ is a saturated or unsaturated, substituted or unsubstituted aliphatic hydrocarbon-based unit containing from 1 to 36 carbon atoms, for example, from 3 to 24 carbon atoms, such as, from 4 to 24 carbon atoms, and

RCOO corresponds to an aliphatic or aromatic monocarboxylic acid RCOOH. For Example, RCOO may be chosen from:

a monocarboxylic acid residue, such as an acid of formula (R₅R₆R₇C)COO in which R₅, R₆ and R₇ are independently chosen from methyl, ethyl, propyl and isopropyl groups; and

an aromatic acid residue comprising a benzenyl ring that is optionally substituted with OH or NH₂ or methyl or ethyl groups.

Non-limiting examples of aliphatic monocarboxylic acids that are suitable for preparing the alkoxylated ester include those containing from 4 to 24 carbon atoms, including for example, those containing from 4 to 18 carbon atoms. Non-limiting examples of aliphatic monocarboxylic acids include 2-ethylhexanoic acid, caproic acid, neopentanoic acid, isostearic acid, neoheptanoic acid and oleic acid.

Non-limiting examples of aromatic monocarboxylic acids include benzoic acid and p-aminobenzoic acid.

The fatty alcohols used to prepare the alkoxylated esters may be saturated or unsaturated, substituted or unsubstituted, aliphatic or aromatic, and may have a straight chain or a branched chain. They may contain from 6 to 24 carbon atoms. In a non-limiting embodiment of the present disclosure, the fatty alcohols contain from 12 to 14 carbon atoms.

As used herein, the term “fatty alcohol” means an aliphatic alcohol containing at least three carbon atoms. Non-limiting examples of fatty alcohols suitable for use in the present disclosure include those containing carbon, hydrogen and oxygen atoms. Further, the fatty alcohol may be saturated or may comprise at least one carbon-carbon double bond.

In a non-limiting embodiment, the fatty alcohol may be chosen from those obtained via the hydrolysis of fats or of plant or animal oils.

Non-limiting examples of the esters of a monocarboxylic acid and of a polypropoxylated fatty alcohol include PPG-3 myristyl ether neoheptanoate sold under the reference Trivasperse, PPG-4 butyloctyl ether ethylhexanoate, and mixtures thereof. These esters may be prepared according to the teaching of United States Patent Application Publication No. 2002/0 192 249, the content of which is herein incorporated by reference.

Alkoxylated Mixed Polyesters

In a non-limiting embodiment of the present disclosure, the alkoxylated ester may be chosen from mixed esters of an alkoxylated alcohol and of a monohydric alcohol with polycarboxylic acids, such as dicarboxylic acids. For example, the alkoxylated ester may be chosen from mixed esters of a polyalkoxylated fatty alcohol and of a monohydric fatty alcohol with dicarboxylic fatty acids.

As used herein, the term “mixed ester” means an ester obtained by reacting a polycarboxylic acid with at least two different alcohols.

As used herein, the term “fatty acid” means an aliphatic carboxylic acid containing at least three carbon atoms. Non-limiting examples of fatty acids that are suitable for use in the present disclosure include those containing carbon, hydrogen and oxygen atoms. These fatty acids may be saturated and/or may comprise at least one carbon-carbon double bond.

In a non-limiting embodiment of the present disclosure, the fatty acid may be chosen from carboxylic acids obtained by the hydrolysis of fats or of plant or animal oils.

Examples of the mixed ester include, but are not limited to, compounds having the following structural formula: 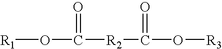
in which R₁ has the structural formula: 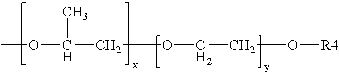
in which:

R₄ is a saturated or unsaturated, substituted or unsubstituted aliphatic unit containing from 4 to 24 carbon atoms;

x is an integer from 3 to 30; y is an integer from 3 to 30;

R₂ is a saturated or unsaturated, substituted or unsubstituted aliphatic unit containing from 4 to 40 carbon atoms; and

R₃ is a saturated or unsaturated, straight-chain or branched-chain aliphatic unit containing from 4 to 32 carbon atoms, for example, from 12 to 24 carbon atoms.

Non-limiting examples of compounds corresponding to the above general formula include:

octyldodecyl PPG-3 myristyl ether dilinoleate, sold under the reference Liquiwax polyEFA by the company Arch Chemical, and having the formula:

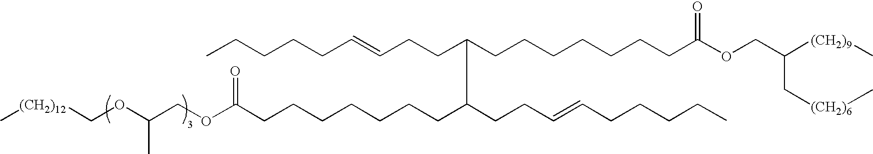

stearyl PPG-3 myristyl ether dilinoleate, sold under the reference Liquiwax polylPL by the company Arch Chemical, and

isostearyl PPG-4 butyloctyl ether dilinoleate.

These mixed esters may be produced by reacting alkoxylated fatty alcohols and monohydric fatty alcohols with dicarboxylic fatty acids.

In a non-limiting embodiment of the present disclosure, the alkoxylated fatty alcohols may be chosen from propoxylated fatty alcohols having a carbon chain length ranging from 4 to 24 carbon atoms and a degree of propoxylation ranging from 3 to 30, for example, from 3 to 15 propylene oxide units. Non-limiting examples of propoxylated fatty alcohols include myristyl alcohol and butyloctanol.

The dicarboxylic acid contains at least two carboxylic groups per molecule. For example, the dicarboxylic acid may be represented by formula (I) below:
HOOC—(CH₂)ₙ—COOH (I)
in which n is an integer from 1 to 16, for example, from 3 to 16.

Non-limiting examples of dicarboxylic acids that are suitable for use in the present disclosure include malonic acid, succinic acid, glutaric acid, adipic acid, pimelic acid, suberic acid, azelaic acid, sebacic acid, 1,9-nonamethylenedicarboxylic acid, 1,10-decamethylenedicarboxylic acid, 1,11-undecamethylenedicarboxylic acid, 1,12-dodecamethylenedicarboxylic acid, 1,13-tridecamethylenedicarboxylic acid, 1,14-tetradeca-methylenedicarboxylic acid, 1,1 5-pentadecamethylenedicarboxylic acid, 1,16-hexadecamethylenedicarboxylic acid, and mixtures thereof. Further, the dicarboxylic acid may also be a diacid dimer. As used herein, the term “diacid dimer” denotes a diacid obtained via an intermolecular polymerization, for example, dimerization, reaction of at least one unsaturated monocarboxylic acid. For example, these diacid dimers may be derived from the dimerization of an unsaturated fatty acid, such as an unsaturated C₈ to C₃₄ fatty acid, for example, a C₁₂ to C₂₂ fatty acid, such as a C₁₆ to C₂₀ fatty acid, for example a C₁₈fatty acid.

Examples of these unsaturated fatty acids include, but are not limited to undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid, arachidonic acid, and mixtures thereof.

In a non-limiting embodiment of the present disclosure, the diacid dimer from which the diol dimer to be esterified is also derived. The diacid dimer, may, for example, be obtained by the dimerization of linoleic acid, optionally followed by hydrogenation of the carbon-carbon bonds. According to one embodiment, the diacid dimer may be in saturated form, i.e. it may comprise no carbon-carbon double bonds. According to another non-limiting embodiment, the possible carbon-carbon double bonds in the diacid dimer may be completely or partially hydrogenated, after an esterification reaction of the diacid dimer with the diol dimer.

In another non-limiting embodiment of the present disclosure, the diacid dimer may be a known product comprising a dicarboxylic acid containing about 36 carbon atoms. This product may also contain a trimeric acid and a monomeric acid, in proportions that depend on the degree of purity of the product. Products whose content of diacid dimer is greater than 70% and others whose content of diacid dimer has been adjusted to 90% or more are known.

Diacid dimers, such as dilinoleic diacids whose stability towards oxidation has been improved by hydrogenation of the double bonds remaining after the dimerization reaction are also known.

In a non-limiting embodiment of the present disclosure, the diacid dimer is chosen from commercially available diacid dimers.

Non-limiting examples of suitable monohydric fatty alcohols that may be used in the present disclosure include those having a carbon chain length ranging from 12 to 24. For example, the monohydric fatty alcohols may be chosen from are octyldodecanol and isostearyl alcohol.

Non-limiting examples of the preparation of the aforementioned esters are provided in International Patent Application Publication No. WO 2004/052 076, the content of which is herein incorporated by reference.

Alkoxylated Polyesters

In another non-limiting embodiment of the present disclosure, the alkoxylated ester may be obtained by the esterification of a polycarboxylic acid with at least two identical or different alkoxylated alcohols, so as to form an ester.

Examples of alkoxylated esters include, but are not limited to esters of formula: 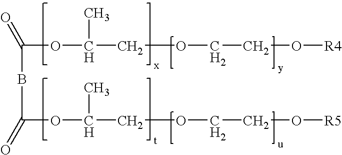
in which:

—OOC—B—COO— is a saturated or unsaturated, substituted or unsubstituted dicarboxylic acid residue as described above, containing from 2 to 40 carbon atoms, wherein B is a linking group containing up to 38 carbon atoms or a bond,

x and y are, independently, integers ranging from 0 to 40, and the sum of x and y ranges from 1 and 80, for example, from 2 to 80,

t and u are, independently, integers ranging from 0 to 40, and the sum of t and u ranges from 1 to 80, for example, from 2 to 80, and

R₄ and R₅ are independently chosen from aliphatic or aromatic, saturated or unsaturated, substituted or unsubstituted hydrocarbon-based units containing from 4 to 36 carbon atoms.

In a non-limiting embodiment of the present disclosure, R₄ and R₅ may be identical or different, may contain from 10 to 22 carbon atoms, may be saturated or unsaturated, and may be substituted or unsubstituted.

In a non-limiting embodiment of the present disclosure, y is an integer ranging from 1 to 40 and x is an integer ranging from 0 to 30, wherein y is greater than x, and if x is equal to 0, y is greater than or equal to 2.

In another non-limiting embodiment of the present disclosure, u is an integer ranging from 1 to 40 and t is an integer ranging from 0 to 30, wherein u is greater than t, and if t is equal to 0, u is greater than or equal to 2.

The dicarboxylic acid may, for example, be chosen from aliphatic or aromatic dicarboxylic acids. In a non-limiting embodiment of the present disclosure, the dicarboxylic acid is chosen from aliphatic carboxylic acids. These aliphatic carboxylic acids may contain, for example, from 2 to 36 carbon atoms. In a non-limiting embodiment, the aliphatic dicarboxylic acids contain from 3 to 8 carbon atoms. Examples of suitable aliphatic dicarboxylic acids include, but are not limited to adipic acid, sebacic acid, malonic acid, succinic acid and maleic acid.

In another non-limiting embodiment of the present disclosure, the dicarboxylic acid is chosen from aromatic dicarboxylic acids. These aromatic dicarboxylic acids may contain, from 8 to 36, for example, from 8 to 12 carbon atoms. Non-limiting examples of suitable aromatic dicarboxylic acids include phthalic acid. In a non-limiting embodiment, the aromatic dicarboxylic acid is 1,2-phthalic acid, which has the lowest melting point of the phthalic acid isomers.

In a non-limiting embodiment of the present disclosure, x and y may each be equal to 15, wherein the total of x and y does not exceed 25. Further, u and t may each be equal to 15, wherein the total of u and t does not exceed 25.

In a non-limiting embodiment of the present disclosure, y or u is greater than or equal to 1, and x or t is greater than or equal to 0. Further, the number of ethoxy units may be greater than the number of propoxy units.

The alkoxylated esters may be prepared according to methods described in International Patent Application Publication No. WO 00/19972, the content of which is herein incorporated by reference.

In a non-limiting embodiment of the present disclosure, R₄ and R₅ are chosen from saturated aliphatic groups. These saturated aliphatic groups may contain, for example, from 14 to 18, such as from 14 to 16, carbon atoms. In a non-limiting embodiment, the saturated aliphatic group(s) is/are myristyl groups having 14 carbon atoms.

In another non-limiting embodiment, when R₄ and R₅ are a myristyl group, y and u may be equal to zero and x and t, which may be the same or different, are integers ranging from 2 to 40 inclusive. For example, the product sold under the reference Cromollient DP3A may be utilized, in which R₄ and R₅ of the above formula are both a myristyl group, —OOC—B—COO— is an adipate, y and u equal 0 and x and t equal 3.

In a non-limiting embodiment of the present disclosure, the alkoxylated ester is present in the composition in an amount ranging from about 1% to about 99%, for example from about 2% to about 60%, such as from about 5% to about 40%, further from about 10% to about 35% by weight, relative to the total weight of the composition.

Hydrocarbon-Based Ester

As used herein, the term “hydrocarbon-based ester” means a compound that is different from the alkoxylated ester described above and comprising at least one ester function COO. According to the present disclosure, the word ester means a monoester, a diester, a triester and, more generally, a polyester that is different from the polyester described above.

In a non-limiting embodiment of the present disclosure, the hydrocarbon-based ester is chosen from oils, including, but not limited to fatty substances that are liquid at atmospheric pressure and at a temperature of 23° C.

The hydrocarbon-based ester may be linear, branched or cyclic, and saturated or unsaturated.

In a non-limiting embodiment of the present disclosure, the hydrocarbon-based ester is a non-volatile oil.

As used herein, the term “volatile oil” means an oil (or non-aqueous medium) capable of evaporating on contact with the skin in less than one hour, at room temperature and at atmospheric pressure. For example, the volatile oil may be a volatile cosmetic oil that is liquid at room temperature, including those volatile oils having a non-zero vapor pressure at room temperature and atmospheric pressure. For example, the volatile oil may be chosen from those volatile oils having a vapor pressure ranging from 0.13 Pa to 40,000 Pa (10⁻³ to 300 mmHg), from 1.3 Pa to 13,000 Pa (0.01 to 100 mmHg), and from 1.3 Pa to 1300 Pa (0.01 to 10 mmHg).

In a non-limiting embodiment of the present disclosure, the hydrocarbon-based ester may correspond to the formula RCOOR′ in which RCOO is chosen from carboxylic acid residues containing from 2 to 30 carbon atoms, and R′ is chosen from hydrocarbon-based chains containing from 1 to 30 carbon atoms.

RCOO may be chosen from aliphatic carboxylic acid residues containing from 2 to 30, such as 4 to 26, and further such as 4 to 22 carbon atoms. Radical R may be chosen from alkyl or alkenyl radicals. As used herein, the term “alkyl radical” means a saturated, linear or branched aliphatic radical of carbon and hydrogen. Further, the term “alkenyl radical,” as used herein, means a linear or branched aliphatic radical of carbon and hydrogen, which is unsaturated, i.e. comprising at least one carbon-carbon double bond. Non-limiting examples of alkenyl radicals that are suitable for use in the present disclosure include those chosen from alkenyl radicals having one, two, and three double bonds.

RCOO may be chosen from fatty acid residues, such as an acid obtained by the hydrolysis of a fatty substance of plant or animal origin.

The radical OR′ may be chosen from saturated or unsaturated, linear or branched alcohol residues, including aliphatic alcohol residues. The radical R′ may be chosen from alkyl radicals and alkenyl radicals, independently of the choice of the radical R, wherein alkyl and alkenyl are defined as above.

OR′ may be chosen from fatty alcohol residues, for example, alcohols obtained by the hydrogenation of a fatty acid, as defined above.

In a non-limiting embodiment of the present disclosure, OR′ may be chosen from aliphatic alcohol residues containing from 2 to 30, such as 4 to 26, and further such as 4 to 22 carbon atoms.

R and R′ are chosen independently of each other. In a non-limiting embodiment, R and R′ are chosen such that they are each both saturated and branched, or both linear and monounsaturated.

The ester may contain up to 60 carbon atoms. In a non-limiting embodiment of the present disclosure, the ester contains from 10 to 45, for example, from 18 to 40, carbon atoms.

In a non-limiting embodiment of the present disclosure, the hydrocarbon-based ester is chosen from branched, saturated monoesters. For example, the ester may be a monoester of a branched saturated aliphatic carboxylic acid and of a branched saturated aliphatic alcohol.

In another non-limiting embodiment of the present disclosure, the hydrocarbon-based ester is chosen from monoesters of linear unsaturated fatty monoacids and linear unsaturated fatty monoalcohols comprising a carbon-carbon double bond.

Examples of esters according to the present disclosure include, but are not limited to neopentanoic acid esters, such as isodecyl neopentanoate, isotridecyl neopentanoate, isostearyl neopentanoate and 2-octyidodecyl neopentanoate, isononanoic acid esters, such as isononyl isononanoate, octyl isononanoate, isodecyl isononanoate, isotridecyl isononanoate, isostearyl isononanoate, isopropyl alcohol esters, such as isopropyl myristate, isopropyl palmitate, isopropyl stearate or isopropyl isostearate, cetyl octanoate, tridecyl octanoate, 2-ethylhexyl 4-diheptanoate, 2-ethylhexyl palmitate, alkyl benzoate, polyethylene glycol diheptanoate, propylene glycol 2-diethyl hexanoate, and mixtures thereof. The ester may also be chosen from synthetic esters, including those of fatty acid, such as purcellin oil, isopropyl myristate, ethyl palmitate, octyl stearate, hydroxylated esters, such as isostearyl lactate, octyl hydroxystearate, diisopropyl adipate, fatty alcohol heptanoates, octanoates, decanoates, and mixtures thereof.

In a non-limiting embodiment, the ester is chosen from isononyl isononanoate, oleyl erucate, 2-octyldodecyl neopentanoate and mixtures thereof.

These hydrocarbon-based ester(s) may be present in the composition in an amount ranging from 5% to 90%, for example, from 10% to 60%, more specifically from 20% to 50% by weight, relative to the total weight of the composition.

Staying Power

The composition of the present disclosure is capable of forming a deposit with a staying power index of greater than or equal to 30%. In a non-limiting embodiment, the composition of the present disclosure is capable of forming a deposit having a staying power index of greater than or equal to 40%. In a further non-limiting embodiment, the composition of the present disclosure is capable of forming a deposit having a staying power index of greater than or equal to 45%. In yet another non-limiting embodiment, the composition of the present disclosure is capable of forming a deposit having a staying power index of greater than or equal to 50%.

In a non-limiting embodiment of the present disclosure, the hydrocarbon-based ester is chosen such that when it is in a sufficient amount in the composition, the composition is capable of forming a deposit having a staying power index greater than or equal to 30%.

The staying power index of the deposit obtained with the composition according to the present disclosure is determined according to the measuring protocol described below.

A support (40 mm x 70 mm rectangle) of an acrylic coating (hypoallergenic acrylic adhesive on polyethylene film sold under the name Blenderme ref. FH5000-55113 by the company 3M Santé) bonded onto a layer of polyethylene foam that is adhesive on the side opposite the one to which the adhesive plaster is fixed (foam layer sold under the name RE40X70EP3 from the company Joint Technique Lyonnais Ind.) is prepared.

The color L*0a*0b*0 of the support, on the acrylic coating side, is measured using a Minolta CR300 calorimeter.

The support thus prepared is preheated on a hotplate maintained at a temperature of 40° C. so that the surface of the support is maintained at a temperature of 33° C.±1° C.

While leaving the support on the hotplate, the composition is applied to the entire non-adhesive surface of the support (i.e. to the surface of the acrylic coating), and is spread out with a brush. A deposit of the composition of about 15 μm is obtained. This deposit is left to dry for 10 minutes.

After drying, the color L*a*b* of the film thus obtained is measured.

The color difference ΔE1 between the color of the film relative to the color of the naked support is then determined via the following relationship:
ΔE1=\sqrt{(L*−L₀*)²+(a*−a₀*)²+(b*−b₀*)²}

The support is then bonded via its adhesive face (adhesive face of the foam layer) to an anvil 20 mm in diameter and equipped with a screw pitch. A sample of the support/deposit assembly is then cut out using a sample punch 18 mm in diameter. The anvil is then screwed onto a press (Statif Manuel Imada SV-2 from the company Someco) equipped with a tensile testing machine (Imada DPS-20 from the company Someco).

A strip 33 mm wide and 29.7 cm long is drawn on a sheet of white photocopier paper with a basis weight of 80 g/m², a first line is marked 2 cm from the edge of the sheet, and a second line is then marked 5 cm from the edge of the sheet, the first and second lines delimited a box on the strip. Next, a first mark and a second mark located in the strip at reference points 8 cm and 16 cm, respectively, from the second mark, are applied. 20 μl of water is then placed on the first mark and 10 μl of refined sunflower oil (sold by the company Lesieur) is then placed on the second mark.

The white paper is placed on the base of the press and the sample placed on the box of the strip of paper is then pressed at a pressure of about 300 g/cm² for 30 seconds. The press is then opened and the sample is again placed just after the second mark (i.e. next to the box). A pressure of about 300 g/cm² is again applied, and the paper is displaced, in a rectilinear manner as soon as the contact is made, at a speed of 1 cm/s over the entire length of the strip such that the sample passes through the water and the oil deposits.

After the sample is removed, some of the deposit has transferred onto the paper. The color L*′, a*′, b*′ of the deposit remaining on the sample is then measured.

The color difference ΔE2 between the color of the deposit remaining on the sample relative to the color of the naked support is then determined via the following relationship
ΔE2=\sqrt{(L*′−L₀*)²+(a*′−a₀*)²+(b*′−b₀*)²}

The transfer index of the composition, expressed as a percentage, is calculated by the following relationship:
100×ΔE2/ΔE1

The measurement is performed on six supports in succession and the transfer index corresponds to the mean of the six measurements obtained with the six supports.

Pasty Substance

The composition of the present disclosure may further contain at least one pasty compound.

The composition of the present disclosure is optionally free of lanolin or of lanolin derivatives.

In a non-limiting embodiment of the present disclosure, the composition comprises an alkoxylated alcohol ester and at least one hydrocarbon-based ester different from said the alkoxylated alcohol ester, wherein said composition is free of lanolin or derivatives thereof.

Examples of lanolin derivatives that are typically used in cosmetic compositions include, but are not limited to liquid lanolin, reduced lanolin, adsorption-purified lanolin, acetylated lanolin, oxypropylenated (5 PO) lanolin wax, liquid lanolin acetate, hydroxylanolin, polyoxyethylene-lanolin, lanolin fatty acid, hard lanolin fatty acid, cholesteryl esters of lanolin fatty acid, lanolin alcohol, lanolic alcohol acetate, isopropyl lanolate, and the like.

Lanolins have the drawback of being sensitive to heat and to ultraviolet light. Further, they have a tendency to become oxidized with evolution of an unpleasant odor, and their strong yellow color prevents them from being used in unpigmented care bases and colorless bases, which limits their use in cosmetic compositions.

The inventors have found that the alkoxylated esters described above may be used as a substitute for lanolin or derivatives thereof.

As used herein, the term “pasty substance” refers to a lipophilic fatty compound, with a reversible solid/liquid change of state, comprising at a temperature of 23° C. a liquid fraction and a solid fraction. In addition, the term “pasty substance” can refer to polyvinyl laurate.

The pasty compound may be chosen from, for example:
lanolin and its derivatives,
polymeric or non-polymeric fluoro compounds,
polymeric or non-polymeric silicone compounds,
vinyl polymers, such as:
   olefin homopolymers,
   olefin copolymers,
   hydrogenated diene homopolymers and copolymers,
   homopolymeric or copolymeric linear or branched oligomers of alkyl(meth)acrylates, including those containing at least one C₈-C₃₀ alkyl group,
   homopolymeric and copolymeric oligomers of vinyl esters containing at least one C₈-C₃₀ alkyl groups,
   homopolymeric and copolymeric oligomers of vinyl ethers containing at least one C₈-C₃₀ alkyl groups,
   liposoluble polyethers resulting from polyetherification between at least one C₂-C₁₀₀ diol, for example, at least one C₂-C₅₀ diol,
   esters,
   and mixtures thereof.

Non-limiting examples of liposoluble polyethers that may be utilized in accordance with the present disclosure include copolymers of ethylene oxide and/or of propylene oxide with at least one C₆-C₃₀ long-chain alkylene oxide, including those wherein the weight ratio of ethylene oxide and/or of propylene oxide to the alkylene oxides ranges from 5:95 to 70:30. In this family, non-limiting mention may be made of copolymers wherein the long-chain alkylene oxides are arranged in blocks with a mean molecular weight ranging from 1000 to 10,000, such as polyoxyethylene/polydodecyl glycol block copolymers, for example, the ethers of dodecanediol (22 mol), and polyethylene glycol (45 EO) sold under the brand name Elfacos ST9 by Akzo Nobel.

Non-limiting examples of suitable pasty esters that may be used in accordance with the present disclosure include:
esters of at least one oligomeric glycerol, such as diglycerol esters, for example, the condensates of adipic acid and of glycerol, for which some of the hydroxyl groups of the glycerols have reacted with a mixture of fatty acids such as stearic acid, capric acid, stearic acid and isostearic acid, and 12-hydroxystearic acid, such as those sold under the brand name Softisan 649 by the company Sasol,
the arachidyl propionate sold under the brand name Waxenol 801 by Alzo,
phytosterol esters,
non-crosslinked polyesters resulting from the polycondensation of at least one linear or branched C₄-C₅₀ dicarboxylic or polycarboxylic acid and at least one C₂-C₅₀ diol or polyol, other than the polyester described above,
aliphatic esters of an ester resulting from the esterification of an ester of an aliphatic hydroxycarboxylic acid with an aliphatic monocarboxylic acid; and mixtures thereof, such as:
   the ester resulting from the esterification reaction of hydrogenated castor oil with isostearic acid in proportions of 1 to 1 (1/1), i.e., hydrogenated castor oil monoisostearate,
   the ester resulting from the esterification reaction of hydrogenated castor oil with isostearic acid in proportions of 1 to 2 (1/2), i.e., hydrogenated castor oil diisostearate,
   the ester resulting from the esterification reaction of hydrogenated castor oil with isostearic acid in proportions of 1 to 3 (1/3), i.e., hydrogenated castor oil triisostearate,
   and mixtures thereof.

A non-limiting example of a pasty compound of plant origin that is suitable for use in the present disclosure is a mixture of soybean sterols and of oxyethylenated (5 EO) oxypropylenated (5 PO) pentaerythritol, sold under the reference Lanolide by the company Vevy.

The pasty compound may be present in the composition of the present disclosure in an amount ranging from 1% to 99%, such as from 1% to 60%, for example, from 2% to 30%, further such as from 5% to 15% by weight, relative to the total weight of the composition.

Dye

The composition of the present disclosure may also comprise at least one dyestuff chosen from dyes, pigments, nacres, and mixtures thereof. This dyestuff may be present in the composition in an amount ranging from 0.001% to 98%, for example, from 0.5% to 85%, further for example, from 1% to 60% by weight, relative to the total weight of the composition.

The dyes may be chosen from liposoluble dyes and water-soluble dyes. Non-limiting examples of liposoluble dyes that are suitable for use in the present disclosure include Sudan red, D & C Red 17, D & C Green 6, β-carotene, soybean oil, Sudan brown, D & C Yellow 11, D & C Violet 2, D & C Orange 5, quinoline yellow and annatto. According to one embodiment, these dyes may be present in the composition in an amount ranging from 0 to 20%, for example from 0.1% to 6%, by weight, relative to the total weight of the composition. Non-limiting examples of water-soluble dyes that may be used include beetroot juice and methylene blue. According to one embodiment, the water soluble dyes, if present, may be present in the composition in an amount ranging from 0.1% to 6% by weight of the composition.

When the composition is in paste or cast form, such as in the form of a lipstick or body makeup product, the dyestuff may be present in the composition in an amount ranging from 0.5% to 50%, for example, from 2% to 40%, further for example, from 5% to 30% by weight, relative to the total weight of the composition.

As used herein the term “pigments” refers to white or colored, mineral or organic particles that are insoluble in the oil(s) of the composition and which are intended to color and/or opacify the composition. Further, the term “fillers” refers to colorless or white, mineral or synthetic, lamellar or non-lamellar particles. The term “nacres,” as used herein refers to iridescent particles, including, but not limited to those produced by certain molluscs in their shell and those that are alternatively synthesized. These fillers and nacres may be utilized, for example, to modify the texture of the composition.

The pigments may be present in the composition in an amount ranging from 0.05% to 30%, for example, from 2% to 20% by weight, relative to the total weight of the composition. Non-limiting examples of mineral pigments that may be utilized in the present disclosure, include titanium oxide, zirconium oxide, cerium oxide, zinc oxide, iron oxide, chromium oxide and ferric blue. Among the organic pigments that may be used in the present disclosure, non-limiting mention may be made of carbon black and barium, strontium, calcium (D & C Red No. 7) and aluminium lakes.

The nacres may be present in the composition in an amount ranging from 0.001% to 20%, for example, from 1% to 15% by weight, relative to the total weight of the composition. Non-limiting examples of nacres that may be utilized in the present disclosure include mica coated with titanium oxide, mica coated with iron oxide, mica coated with a natural pigment, and mica coated with bismuth oxychloride, such as colored titanium mica.

The composition may further comprise goniochromatic pigments, such as multilayer interference pigments, and/or reflective pigments. These two types of pigment are described in French Patent Application No. FR 02/09246, the content of which is herein incorporated by reference.

Fillers

The composition of the present disclosure may further contain at least one filler in an amount ranging from 0.001% to 35%, for example, from 0.5% to 15% by weight, relative to the total weight of the composition.

Non-limiting examples of fillers that may be used in the present invention include:
talc, mica, kaolin or starch
Nylon® powders, such as Orgasol
polyethylene powders
polytetrafluoroethylene, such as Teflon® powders
boron nitride
copolymer microspheres, such as Expancel® from Nobel Industrie
Polytrap® 603 from Dow Corning
Polypore® L 200 from Chemdal Corporation
silicone resin microbeads, such as Tospearl® from Toshiba
silica-based fillers, such as Aerosil 200, Aerosil 300; Sunsphere L-31 and Sunsphere H-31 sold by Asahi Glass; Chemicelen sold by Asahi Chemical; composites of silica and of titanium dioxide, such as the TSG series sold by Nippon Sheet Glass
polyurethane powders, such as powders of crosslinked polyurethane comprising a copolymer, wherein said copolymer comprises trimethylol hexyllactone. For example, the copolymer may be a polymer of hexamethylene diisocyanate/trimethylol hexyllactone. Non-limiting examples of such particles include Plastic Powder D-400® and Plastic Powder D-800® from the company Toshiki.

The filler may exhibit a mean particle size of less than or equal to 100 μm, for example, from 1 to 50 μm, more specifically, from 4 to 20 μm.

The filler may be in any form, including spherical or platelet form.

Wax

The composition of the present disclosure may also contain at least one wax. For the purposes of the present disclosure, the term “wax” means a lipophilic fatty compound, which is solid at room temperature (25° C.), with a reversible solid/liquid change of state, having a melting point of greater than 30° C. that may be up to 200° C., a hardness of greater than 0.5 MPa, and having in the solid state an anisotropic crystal organization. By bringing a wax to its melting point, it is possible to make it miscible with oils and to form a microscopically homogeneous mixture, but on returning the temperature of the mixture to room temperature, recrystallization of the wax in the oils of the mixture is obtained.

Non-limiting examples of waxes that may be used in the present disclosure include compounds that are solid at room temperature and which are intended to structure the composition, for example, in stick form. These waxes may be chosen from, for example, hydrocarbon-based waxes, fluoro waxes and/or silicone waxes and may be of plant, mineral, animal and/or synthetic origin. Further, these waxes may exhibit a melting point of greater than 40° C., for example, greater than 45° C.

Examples of waxes that may be used in the present disclosure include, but are not limited to those waxes that are generally used in cosmetics, for example: waxes of natural origin, such as beeswax, carnauba wax, candelilla wax, ouricoury wax, Japan wax, cork fiber wax, sugarcane wax, rice wax, montan wax, paraffin, lignite wax, microcrystalline wax, ceresin and ozokerite, and hydrogenated oils, such as jojoba oil; synthetic waxes, such as polyethylene waxes derived from the polymerization or copolymerization of ethylene and Fischer-Tropsch waxes, fatty acid esters, such as octacosanyl stearate, glycerides that are solid at 40° C., more specifically 45° C., silicone waxes, such as alkyl or alkoxy dimethicones containing an alkyl or alkoxy chain of 10 to 45 carbon atoms, poly(di)methylsiloxane esters that are solid at 40° C., the ester chain of which contains at least 10 carbon atoms; and mixtures thereof.

In a non-limiting embodiment, the composition according to the present disclosure contains a polyethylene wax with a weight-average molecular mass of between 300 and 700, for example a polyethylene wax with a weight-average molecular mass equal to 500 g/mol.

The wax may be present in the composition of the present disclosure in an an amount ranging from, for example, 0.01% to 50%, further for example, from 2% to 40%, such as from 5% to 30% by weight, relative to the total weight of the composition.

Non-Volatile Oil

The composition of the present disclosure may further comprise at least one non-volatile oil other than the alkoxylated alcohol ester and other than the apolar oil described above. The non-volatile oil may be chosen from, for example:
hydrocarbon-based oils of animal origin, such as perhydrosqualene;
hydrocarbon-based plant oils such as liquid triglycerides of fatty acids containing from 4 to 10 carbon atoms, such as heptanoic or octanoic acid triglycerides and jojoba oil;
linear or branched hydrocarbons of mineral or synthetic origin, such as liquid paraffins and derivatives thereof, and petroleum jelly;
fatty alcohols containing from 12 to 26 carbon atoms, such as octyldodecanol, 2-butyloctanol, 2-hexyldecanol, 2-undecylpentadecanol and oleyl alcohol;
fluoro oils that are optionally partially hydrocarbon-based and/or silicone-based;
silicone oils, such as volatile or non-volatile, linear or cyclic polydimethylsiloxanes (PDMS); polydimethylsiloxanes comprising groups chosen from alkyl, alkoxy and phenyl groups that are pendent or at the end of a silicone chain, these groups containing from 2 to 24 carbon atoms; phenyl silicones, such as phenyl trimethicones, for example, the phenyl trimethicone sold under the trade name DC556 by Dow Corning, phenyl dimethicones, phenyltrimethylsiloxydiphenylsiloxanes, diphenyl dimethicones, diphenylmethyldiphenyltrisiloxanes, and 2-phenylethyl trimethylsiloxysilicates;
fatty acids containing from 12 to 26 carbon atoms, such as oleic acid;
and mixtures thereof.
   Non-Volatile Oil of High Molecular Mass

According to one embodiment, the composition of the present disclosure may further comprise a non-volatile oil of high molecular mass, for example, from 650 to 10,000 g/mol.

Further, the composition according to the present disclosure may comprise at least one oil having a molar mass ranging from 650 to 10,000 g/mol, for example from 900 to 7500 g/mol, in an amount ranging from 2% to 30%, for example, from 5% to 25%, such as from 5% to 15% by weight, relative to the total weight of the composition.

Non-limiting examples of oils having a molecular mass ranging from 650 to 10,000 g/mol that may be utilized in the present disclosure include:

polybutylenes, such as Indopol H-100 (molar mass or MM=965 g/mol), Indopol H-300 (MM=1340 g/mol) and Indopol H-1 500 (MM=2160 g/mol) sold or manufactured by the company Amoco,

hydrogenated polyisobutylenes, such as Panalane H-300 E sold or manufactured by the company Amoco (M=1340 g/mol), Viseal 20000 sold or manufactured by the company Synteal (MM=6000 g/mol) and Rewopal PIB 1000 sold or manufactured by the company Witco (MM=1000 g/mol),

polydecenes and hydrogenated polydecenes, such as Puresyn 150 (MM=9200 g/mol) sold by the company Mobil Chemicals,

vinylpyrrolidone copolymers, such as the vinylpyrrolidone/1-hexadecene copolymer Antaron V-216 sold or manufactured by the company ISP (MM=7300 g/mol),

esters such as:
a) linear fatty acid esters having from 35 to 70 carbons, such as pentaerythrityl tetrapelargonate (MM=697.05 g/mol),
b) hydroxylated esters, such as polyglyceryl-2 triisostearate (MM=965.58 g/mol),
c) aromatic esters, such as tridecyl trimellitate (MM=757.19 g/mol),
d) esters of branched C₂₄-C₂₈ fatty alcohols or fatty acids, such as those described in European Patent Application No. EP-A-0 955 039, triisoarachidyl citrate (MM=1033.76 g/mol), pentaerythrityl tetraisononanoate (MM=697.05 g/mol), glyceryl triisostearate (MM=891.51 g/mol), glyceryl 2-tridecyl tetradecanoate (MM=1143.98 g/mol), pentaerythrityl tetraisostearate (MM=1202.02 g/mol), polyglyceryl-2 tetraisostearate (MM=1232.04 g/mol) and pentaerythrityl 2-tetradecyl tetradecanoate (MM=1538.66 g/mol),
e) diol dimer esters and polyesters, such as esters of at least one diol dimer and of at least one fatty acid, and esters of at least one diol dimer and of at least one diacid, wherein:
   the esters of at least one diol dimer and at least one monocarboxylic acid may be obtained from monocarboxylic acids containing from 4 to 34 carbon atoms, for example, from 10 to 32 carbon atoms, which acids are linear, branched, saturated or unsaturated. Non-limiting examples of monocarboxylic acid that maybe mentioned include fatty acids.
   The esters of diol dimer and of dicarboxylic acid may be obtained from at least one diacid dimer derived from the dimerization of an unsaturated C₈ to C₃₄, for example C₁₂ to C₂₂, further for example, C₁₆ to C₂₀, such as a C₁₈ fatty acid. In a non-limiting embodiment, the diacid dimer from which the diol dimer is to be esterified is also derived.
   The diol dimer esters may be obtained from a diol dimer produced by the catalytic hydrogenation of a diacid dimer as described above, for example hydrogenated dilinoleic diacid. Non-limiting examples of diol dimer esters that may be utilized in the present disclosure include the esters of dilinoleic diacids and of dilinoleyl diol dimers sold by the company Nippon Fine Chemical under the trade names Lusplan DD-DA5® and DD-DA7®,

silicone oils such as phenyl silicones, for example, Belsil PDM 1000 from the company Wacker (MM=9000 g/mol),

oils of plant origin, such as sesame oil (820.6 g/mol),

and mixtures thereof.

The non-volatile oils may be present in the composition in an amount ranging from 0.001% to 90%, for example, from 0.05% to 60%, such as, from 1% to 35% by weight, relative to the total weight of the composition.

Volatile Oil

The composition of the present disclosure may further comprise at least one volatile oil.

As used herein, the term “volatile oil” means an oil (or non-aqueous medium) capable of evaporating on contact with the skin in less than one hour, at room temperature and atmospheric pressure. The volatile oil may be chosen from, for example volatile cosmetic oils that are liquid at room temperature, including those having a non-zero vapor pressure at room temperature and atmospheric pressure, as well as those having a vapor pressure ranging from 0.13 Pa to 40,000 Pa (10⁻³ to 300 mmHg), for example, from 1.3 Pa to 13 000 Pa (0.01 to 100 mmHg), such as, from 1.3 Pa to 1300 Pa (0.1 to 10 mmHg).

In addition, the volatile oil may have a boiling point, measured at atmospheric pressure, ranging from 150° C. to 260° C., for example, from 170° C. to 250° C.

As used herein, the term “hydrocarbon-based oil” means an oil formed from carbon and hydrogen atoms, and which may further include oxygen and nitrogen atoms. The hydrocarbon based oil does not contain silicon or fluorine atoms. The hydrocarbon-based oil may, however, contain ester, ether, amine or amide groups.

The term “silicone oil,” as used herein, refers to oils containing at least one silicon atom, including those containing Si—O groups.

As used herein, the term “fluoro oil” means an oil containing at least one fluorine atom.

The volatile oil may be, for example, chosen from silicone oils and hydrocarbon-based oils.

Silicone oils that may be utilized in the present disclosure include, but are not limited to silicone oils with a flash point ranging from 40° C. to 102° C., for example, those with a flash point of greater than 55° C. and less than or equal to 95° C., such as those with a flashpoint ranging from 65° C. to 95° C.

Non-limiting examples of volatile silicone oils that may be used in the present disclosure, include linear or cyclic silicones having a viscosity at room temperature of less than 8 cSt, including those containing from 2 to 7 silicon atoms, and these silicones optionally comprising alkyl or alkoxy groups containing from 1 to 10 carbon atoms. As volatile silicone oils that may be used in the present disclosure, non-limiting mention may be made of octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane and decamethyltetrasiloxane, and mixtures thereof.

As volatile silicone oils that may be used in the present disclosure, non-limiting mention may be made of the silicones described in the unpublished French Patent Application FR 03/04259.

The volatile hydrocarbon-based oil that may be used in the present disclosure may be chosen from, for example, hydrocarbon-based oils having a flash point ranging from 40° C. to 102° C., for example, from 40° C. to 55° C., such as from 40° C. to 50° C.

Non-limiting examples of volatile hydrocarbon-based oils that may be utilized in the present disclosure include volatile hydrocarbon-based oils containing from 8 to 16 carbon atoms, and mixtures thereof, including branched C₈-C₁₆ alkanes, such as C₈-C₁₆ isoalkanes (also known as isoparaffins), isododecane, isodecane and isohexadecane, the oils sold under the trade names Isopar or Permethyl, and branched C₈-C₁₆ esters, such as isohexyl neopentanoate, and mixtures thereof. In a non-limiting embodiment, the volatile hydrocarbon-based oil is chosen from volatile hydrocarbon-based oils containing from 8 to 16 carbon atoms, and mixtures thereof, such as isododecane, isodecane and isohexadecane. In a non-limiting embodiment, the volatile hydrocarbon-based oil is isododecane.

The volatile oil may be present in the composition in an amount ranging from 5% to 97.5%, for example, from 10% to 75%, such as from 20% to 50%, further such as from 30% to 40%, for example, 35% by weight, relative to the total weight of the composition.

Additives

The composition of the present disclosure may further comprise additional additives that are usually used in cosmetics. Non-limiting examples of these additional additives include water, antioxidants, film-forming polymers, preserving agents, neutralizers, plasticizers, lipophilic gelling agents or liquid non-aqueous compounds, aqueous-phase-gelling agents, dispersants and cosmetic active agents. These additives, with the exception of water, may be present in the composition in an amount ranging from 0 to 70%, for example, from 1% to 50%, such as from 1% to 10% by weight, relative to the total weight of the composition. Water, if present, may be contained in the composition in an amount ranging from 0.0005% to 20%, for example, from 0.001% to 10% by weight, relative to the total weight of the composition.

As cosmetic active agents that may be used in the present disclosure, non-limiting mention may be made of vitamins A, E, C, B₃ and F, provitamins, such as D-panthenol, glycerol, calmatives, such as a-bisabolol, aloe vera, allantoin, plant extracts or essential oils, protecting or restructuring agents, such as ceramides, “refreshing” active agents, such as menthol and its derivatives, emollients (cocoa butter, dimethicone), moisturizers (arginine PCA), anti-wrinkle active agents, essential fatty acids and sunscreens, and mixtures thereof.

Needless to say, a person skilled in the art will take care to select the optional additional additives and/or the amount thereof such that the advantageous properties of the composition according to the present disclosure are not, or are not substantially, adversely affected by the envisaged addition.

Galenical Forms

The compositions according to the present disclosure have a multitude of applications, concerning all colored or uncolored cosmetic products, such as lipsticks.

The composition of the present disclosure may be in the form of a solid, compacted or cast composition, such as a stick or a dish, or in pasty or liquid form. In a non-limiting embodiment, the composition is in solid form, i.e. in hard form (not flowing under its own weight), and may be cast or compacted, for example, as a stick or a dish.

The composition may be in the form of a paste, a solid or a cream. It may be an oil-in-water or water-in-oil emulsion, a solid or soft anhydrous gel, or alternatively in the form of a loose or compacted powder, and can also exist in a two-phase form. When the composition is in the form of a oily phase, optionally an anhydrous continuous phase, it may also contain an aqueous phase in an amount less than 5% by weight, relative to the total weight of the composition.

The composition according to the present disclosure may be in the form of a colored or uncolored skincare composition, in the form of an anti-sun or makeup-removing composition, or alternatively in the form of a hygiene composition. If cosmetic active agents are utilized in the composition, the composition may be used as a non-therapeutic care or treatment base for the skin, such as the hands or the face, or for the lips (lip balms, for protecting the lips against the cold and/or sunlight and/or the wind), or a product for artificially tanning the skin.

The composition of the present disclosure may be in the form of a colored skin makeup product, such as for the face, i.e. as a blusher, a makeup rouge, an eyeshadow, a body makeup product, such as a semi-permanent tattoo product, a lip makeup product, such as a lipstick or a lip gloss, possibly having non-therapeutic care or treatment properties, a makeup product for the integuments, such as a nail varnish, a mascara, an eyeliner, or a hair coloring or haircare product.

In one non-limiting embodiment of the present disclosure, the composition is in the form of a lipstick or a lip gloss.

In another non-limiting embodiment, the composition of the present disclosure is physiologically and/or cosmetically acceptable, i.e. it is non-toxic and able to be applied to human skin, integuments and/or lips.

The term “cosmetically acceptable,” as used herein means having a pleasant taste, feel, appearance and/or odor, which may be applied for several days and/or months.

The composition according to the present disclosure may be manufactured via processes generally used in cosmetics.

Other than in the operating examples, or where otherwise indicated, all numbers expressing quantities of ingredients, reaction conditions, and so forth in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches. Also, where a range is given, even if the term “between” is used, the ranges defined include the stated endpoints.

Notwithstanding the numerical ranges and parameters setting forth the broad scope of the invention as approximations, the numerical values set forth in the specific examples are reported as precisely as possible. Any numerical value, however, inherently contains certain errors necessarily resulting from the standard deviation found in its respective testing measurement.

The examples that follow serve to illustrate the invention without, however, being limiting in nature.

### EXAMPLE 1

### Stick of Lipstick

| | Amount in |
|---|---|
| Chemical name | grams |
| Ethers of dodecanediol and of polyethylene glycol | 10.3 |
| Octyldodecyl PPG-3 myristyl ether dimer dilinoleate | 22.15 |
| (Liquiwax PolyEFA) | |
| Isostearyl neopentanoate | 30.3 |
| Isocetyl stearoyl stearate | 20.6 |
| Polyvinyl laurate | 16.5 |
| Di-tert-butyl 4-hydroxytoluene | 0.15 |
| Vinyl acetate/allyl stearate copolymer | 7.5 |
| Hydrogenated polyisobutene | 10 |
| Polybutene/polyisobutene copolymer | 10 |
| Polyethylene wax | 3.4 |
| Microcrystalline wax | 2.55 |
| Octacosanyl stearate | 4.25 |
| Esters of linear acids and alcohols | 3.4 |
| Alumina/silica/trimethylolpropane-treated rutile titanium oxide | 0.27 |
| Aluminium lake of the disodium salt of phloxin B on alumina, | 0.66 |
| aluminium benzoate | |
| Calcium salt of lithol B red | 0.16 |
| Brown, yellow iron oxides | 0.64 |
| Black iron oxide | 0.66 |
| Polydimethylsiloxane-coated porous silica microspheres | 5 |
| Fragrance | 0.4 |

The mean gloss of this lipstick T0h and T5h was measured at 60° C., according to the above-described method, is 79 out of 100.

### EXAMPLE 2

### Lipstick

| Chemical Name | Weight % |
|---|---|
| Preserving agent | 0.1 |
| Distearyldimethylammonium-modified hectorite | 0.6 |
| Aluminium lake of eosin on alumina (16/84) (Cl: 45380:3 + | 0.6 |
| 77002) and on alumina and TiO₂ (20/80) (Cl: +77891) | |
| Black iron oxide (Cl: 77499) | 0.06 |
| Aluminium lake of Brilliant Yellow FCF on alumina (42/58) | 3.3 |
| (Cl: 15985:1 + 77002) | |
| Microcrystalline wax (C20-C60) | 2.55 |
| Plant isostearic and adipic fatty acid glyceryl esters | 6.01 |
| (Softisan 100) | |
| Microcrystalline wax | 8 |
| Protected arara oil | 12.02 |
| Ethers of dodecanediol (2.2 mol) and of polyethylene glycol | 6.01 |
| (45 EO) (Elfacos ST 9 from Akzo Nobel) | |
| Oleyl erucate | 12.02 |
| Calcium lake of Lithol B Red on colophony and barium | 2.9 |
| sulfate (60/20/20) (Cl: 15850:1 + 77120) | |
| Virgin sesame oil | 15.61 |
| Oxypropylenated (3 PO) myristyl diadipate (Cromollient | 16.95 |
| DP3A from Croda) | |
| Polyglycerolated (3 mol) beeswax | 4.2 |
| Alumina/silica/trimethylolpropane-treated rutile titanium oxide | 1.8 |
| (Cl: 77891) | |
| Dilinoleyl diol dimer/dilinoleic dimer copolymer (Lusplan | 7.27 |
| DD-DA5 from Nippon Fine Chemical) | |

What is claimed is:

1. A cosmetic composition, comprising at least one alkoxylated ester derived from an alkoxylated alcohol and a carboxylic acid, and at least one hydrocarbon-based ester different from said at least one alkoxylated ester, wherein, when said composition forms a deposit, said deposit exhibits a staying power index of greater than or equal to 30%.

2. The cosmetic composition of claim 1, wherein said at least one alkoxylated ester is chosen from:
esters obtained by reacting a monocarboxylic acid with a polyalkoxylated alcohol,
polyesters obtained by reacting a polycarboxylic acid with a stoichiometric excess of at least one alkoxylated alcohol relative to the number of acid functions in the said acid,
polyesters in which only one ester function is obtained by reacting an acid function of a polycarboxylic acid with an alkoxylated alcohol,
polyesters comprising at least one ester function obtained by reacting an acid function of a polycarboxylic acid with an alkoxylated alcohol, and at least one ester function obtained by reacting another acid function of the said polycarboxylic acid with a fatty alcohol, and
mixtures thereof.

3. The cosmetic composition of claim 2, wherein said at least one alkoxylated ester is obtained by reacting a monocarboxylic acid with a polyalkoxylated alcohol.

4. The cosmetic composition of claim 3, wherein said at least one alkoxylated ester is chosen from compounds of the formula: 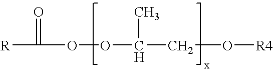
in which:
x is an integer ranging from 2 to 40;
R₄ is chosen from saturated or unsaturated, substituted or unsubstituted aliphatic hydrocarbon-based units containing from 3 to 36 carbon atoms; and
RCOO is chosen from:
a monocarboxylic acid residues; and
aromatic acid residues comprising a benzenyl ring optionally substituted with at least one OH, NH₂, methyl or ethyl group.

5. The cosmetic composition of claim 4, wherein said monocarboxylic acid residue is chosen from acids of the formula (R₅R₆R₇C)COO in which R₅, R₆ and R₇ are independently chosen from methyl, ethyl, propyl and isopropyl groups.

6. The cosmetic composition of claim 4, wherein said monocarboxylic acid residue is chosen from 2-ethylhexanoic acid, caproic acid, neopentanoic acid, isostearic acid, neoheptanoic acid and oleic acid.

7. The cosmetic composition of claim 4, wherein R₄ contains from 4 to 24 carbon atoms.

8. The cosmetic composition of claim 4, wherein x ranges from 3 to 10.

9. The cosmetic composition of claim 3, wherein the ester of aliphatic and aromatic monocarboxylic acids and of polypropoxylated fatty alcohols is chosen from PPG-3 myristyl ether neoheptanoate and PPG-4 butyloctyl ether ethylhexanoate, and mixtures thereof.

10. The cosmetic composition of claim 2, wherein the alkoxylated ester is chosen from the polyesters obtained by reacting at least one polycarboxylic acid with a stoichiometric excess of at least one alkoxylated alcohol relative to the number of acid functions in the acid.

11. The cosmetic composition of claim 10, wherein the alkoxylated alcohol contains an alkyl chain of from 8 to 36 carbon atoms.

12. The cosmetic composition of claim 10, wherein the alkoxylated alcohol contains a myristyl chain.

13. The cosmetic composition of claim 10, wherein the at least one polycarboxylic acid is a dicarboxylic acid and wherein the dicarboxylic acid is aliphatic and contains from 2 to 36 carbon atoms.

14. The cosmetic composition of claim 13, wherein the dicarboxylic acid is chosen from adipic acid, malonic acid, succinic acid, phthalic acid and maleic acid.

15. The cosmetic composition of claim 2, wherein the alkoxylated ester is one of the polyesters in which only one ester function is obtained by reacting an acid function of a polycarboxylic acid with an alkoxylated alcohol.

16. The cosmetic composition of claim 1, wherein the alkoxylated ester is chosen from compounds having the following structural formula: 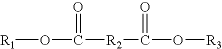
in which R₁ has the structural formula: 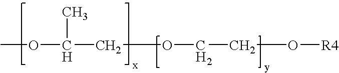
in which:
R₄ is chosen from saturated and unsaturated, substituted and unsubstituted aliphatic units containing from 4 to 24 carbon atoms;
x is an integer ranging from 3 to 30; y is an integer ranging from 3 to 30;
R₂ is chosen from saturated and unsaturated, substituted and unsubstituted aliphatic unit containing from 4 to 40 carbon atoms; and
R₃ is chosen from saturated and unsaturated, straight-chain and branched-chain aliphatic units containing from 4 to 32 carbon atoms.

17. The cosmetic composition of claim 16, wherein R₄ is a saturated aliphatic unit of 12 or 20 carbon atoms.

18. The cosmetic composition of claim 16, wherein R₂ is a saturated aliphatic unit containing from 4 to 40 carbon atoms.

19. The cosmetic composition of claim 16, wherein x and y are, independently of each other, equal to 3 or 4.

20. The cosmetic composition of claim 16, wherein R₃ is a saturated branched-chain aliphatic unit containing from 12 to 20 carbon atoms.

21. The cosmetic composition of claim 20, wherein R₃ is chosen from octyldodecyl, isostearyl and stearyl.

22. The cosmetic composition of claim 16, wherein the alkoxylated ester is octyidodecyl PPG-3 myristyl ether dilinoleate.

23. The cosmetic composition of claim 16, wherein the alkoxylated ester is isostearyl PPG-4 butyloctyl ether dilinoleate.

24. The cosmetic composition of claim 1, wherein the hydrocarbon-based ester is present in a sufficient amount in the composition such that the composition, when formed into a deposit, exhibits a staying power index of greater than or equal to 30%.

25. The cosmetic composition of claim 1, wherein the hydrocarbon-based ester is a monoester.

26. The cosmetic composition of claim 1, wherein the ester is an ester of an aliphatic acid containing from 2 to 28 carbon atoms and of an aliphatic alcohol containing from 1 to 28 carbon atoms.

27. The cosmetic composition of claim 1, wherein the hydrocarbon-based ester is not a volatile oil.

28. The cosmetic composition of claim 1, wherein the hydrocarbon-based ester is chosen from synthetic esters, hydroxylated esters, neopentanoic acid esters, isononanoic acid esters, and isopropyl alcohol esters.

29. The cosmetic composition of claim 24, wherein when the composition forms a deposit, the deposit exhibits a staying power index of greater than or equal to 40%.

30. The cosmetic composition of claim 1, wherein when the composition forms a deposit, the deposit exhibits a staying power index of greater than or equal to 50%.

31. The cosmetic composition of claim 1, further comprising at least one dyestuffs chosen from water-soluble dyes, liposoluble dyes and pulverulent dyestuffs.

32. The cosmetic composition of claim 1, further comprising at least one fatty substance chosen from waxes and pasty fatty substances.

33. The cosmetic composition of claim 1, further comprising at least one cosmetic ingredient chosen from vitamins, thickeners, film-forming agents, gelling agents, trace elements, softeners, sequestering agents, fragrances, acidifying agents, basifying agents, preserving agents, sunscreens, surfactants, antioxidants, fibers, agents for preventing hair loss, eyelash care agents, antidandruff agents and propellants.

34. The cosmetic composition of claim 1, wherein the composition is in a form chosen from suspensions, dispersions, solutions, gels, emulsions, creams, pastes, mousses, dispersion of vesicles, two-phase or multiphase lotions, sprays, powders, sticks and cast solids.

35. The cosmetic composition of claim 1, wherein the composition is in anhydrous form.

36. The cosmetic composition of claim 1, wherein the composition is a makeup or care composition for keratin materials.

37. The cosmetic composition of claim 1, wherein the composition is a lip makeup product.

38. A process for making up and/or caring for the skin or the lips, comprising applying to the skin, the lips and/or the integuments at least one composition comprising at least one alkoxylated ester derived from an alkoxylated alcohol and a carboxylic acid, and at least one hydrocarbon-based ester different from said at least one alkoxylated ester, wherein, when said composition forms a deposit, said deposit exhibits a staying power index of greater than or equal to 30%.
